(19) United States
(12) Patent Application Publication
(10) Pub. No.: US 2005/0188990 A1
(43) Pub. Date: Sep. 1, 2005

(54) Multifunctional integrated filter and breathing conduit

(76) Inventors: Atsuo F. Fukunaga, Palos Verdes Peninsula, CA (US); Alex S. Fukunaga, Palos Verdes Peninsula, CA (US); Blanca M. Fukunaga, Palos Verdes Peninsula, CA (US)

Correspondence Address: DANIEL B. SCHEIN, P. O. BOX 28403, SAN JOSE, CA, 95159, US

(21) Appl. No.: 11/056,665

(22) Filed: Feb. 12, 2005

Related U.S. Application Data
(60) Provisional application No. 60/544,856, filed on Feb. 14, 2004.

(51) Int. Cl. A61M 16/00; A62B 7/00

(52) U.S. Cl. 128/204.18; 128/205.12

(57) ABSTRACT

A device for use in constructing a breathing circuit has at least a first tube and a filter; the filter has a proximal and a distal end; the first tube is attached to the filter distal end. A second tube can be attached to the filter proximal end, wherein the second tube can be detached from the filter for reuse in a breathing circuit formed with the foregoing components. The filter and the first tube may be disposed of after a single use. The first tube has a length sufficient to maintain the filter at a desired distance from a patient airway device when connected thereto. In a preferred embodiment, a fresh gas outlet is provided at the distal end of the first tube resulting in minimal or substantially no mixing space in the circuit, while the disposable first tube and filter create less medical waste than that created by prior art circuits. Mixing space refers to space distal of the fresh gas outlet into the circuit where the fresh gases can mix with recirculated or other gases. In embodiments, the mixing space is less than 15 cm³ or less than about 5 cm³, and the distal disposable filter and tube device is less than 50 cm in length.

### RELATED APPLICATION DATA

This application claims priority of U.S. Provisional application 60/544,856, filed Feb. 14, 2004.

### FIELD OF THE INVENTION

The present invention relates to devices or apparatus for use in resuscitating and/or providing assisted ventilation or anesthesia to patients in a variety of settings, such as in operating rooms, intensive care units, emergency medicine clinics, ambulances, and trauma situations. More specifically, the present invention relates to systems and methods for connecting patients to anesthesia machines, ventilators, breathing mouthpieces and the like. More particularly, the invention relates to filters and breathing circuits comprising a disposable component and a reusable component, which leads to a substantial reduction in medical waste, yet provides a multifunctional and versatile respiratory device that has minimal flow resistance and apparatus dead space.

### BACKGROUND OF THE INVENTION

In respiratory care, a wide range of devices to aid breathing are known. These devices are generally designed for very specific uses. For example, in accidents and trauma situations a patient may be unconscious and not breathing; a mouth to mask resuscitator and/or an air bag, such as an AMBU® bag, may be used in resuscitating the patient and providing breathing support. The patient may breathe sporadically, breathe independently, and/or may shift between such states unpredictably.

Mechanical ventilators or respirators are connected to patients via breathing circuits, which generally comprise tubing for providing and exhausting gases, filters, and other components. Such circuits, filters and other components for use therewith are known to those of skill in the art and described in detail in numerous patents, scientific articles, and product information literature. For example, information on breathing systems, and anesthetic and assisted ventilation techniques can be found in U.S. Pat. Nos. 3,556,097, 3,856,051, 4,007,737, 4,188,946, 4,463,755, 4,232,667, 5,823,184, 5,778,872, Austrian Patent No. 93,941, Dorsch, J. A., and Dorsch, S. E., Understanding Anesthesia Equipment.: Construction, Care and Complications. Williams & Wilkins Co., Baltimore (1974), Andrews, J. J., “Inhaled Anesthetic Delivery Systems,” in Anesthesia, 4^{th} Ed. Miller, Ronald, M. D., Editor, Churchill Livingston, Inc., N.Y. (1986). The text of all documents referenced herein, including documents referenced within referenced documents, is hereby incorporated by reference as if same were reproduced in full below.

U.S. Pat. No. 5,983,891, to Fukunaga et al., discloses a respiratory system providing a filter at the proximal end of a respiratory conduit. The filter is attachable to and detachable from a proximal terminal for fluid connection of the respiratory conduit to an inspiratory gas input and to an expiratory gas outlet in a ventilator or anesthesia machine.

U.S. Pat. No. 5,213,096, to Kihlberg et al., discloses a filter device having a Y-piece that includes a patient attachment tube, and a pair of additional attachment tubes adapted for connection to an inhalation tube and an exhalation tube, respectively. The filter includes a sample withdrawal means for obtaining a gas sample from the apparatus during exhalation.

U.S. Pat. No. 5,284,160, to Dryden, discloses “a sampling adaptor suitable for use in a unilimb breathing system with three or more hoses that includes a breathing hose connector,” with “a filter that encloses the sampling end of a flexible sampling hose, and a patient end connector”.

U.S. Pat. No. 5,195,527, to Hicks, discloses a filter with deflectors that enable reduction of the overall size of the filter to reduce apparatus dead space.

U.S. Pat. No. 4,188,946, to Watson, discloses a filter located between the proximal or machine end connector of a Bain type breathing circuit and a control module, the latter including on O₂ analyzer, adjustable pressure warning and control device, pressure gauge, and manually controlled scavenger valve.

U.S. Pat. No. 6,564,799, to Fukunaga et al., discloses a multilumen filter device for use with a unilimb respiratory circuit that has a housing with first and second filter chambers, wherein each chamber is in fluid communication with respective independent fluid paths extending distally and proximally therefrom. The Fukunaga multilumen filter device can be located at the proximal end of the circuit to connect a ventilator or anesthesia machine to the flexible respiratory conduit running to a patient.

U.S. Pat. No. 4,516,573, to Gedeon, discloses a device for connecting a respirator or an anesthesia machine to a patient comprising a short flexible hose with a conical shape which connects one end to an endotracheal tube and the other to the Y-piece of a circuit connecting the respirator or anesthesia machine. Incorporated in part of the hose is a flexible heat and moisture exchanger (HME) body.

While the above devices disclose disposable filters and/or filter combined with a HME device and/or respirtory conduit(s), none of them comprise a device wherein the fresh gas port or outlet is located near or at the distal terminus of the circuit and distal of a disposable filter, HME or other breathing device, which would minimize fresh gas mixing space.

### Respiratory Circuits and Filters

Filter devices used with breathing circuits are commonly connected either at the distal, patient end of the circuit or at the proximal, machine end of the breathing circuit. For example, a filter at the distal end of a circuit can be connected between the respiratory conduit and an airway device, such as an endotracheal tube, laryngeal tube, laryngeal mask or tracheostomy outlet. Heat and moisture exchange (HME) devices or filter/HME devices may also be used in combination with or in place of a filter. Prior art filters and/or HME devices connected at the patient (i.e., distal) end of a circuit add substantial fresh gas mixing and apparatus dead space, and they are bulky and obstruct the patient's face. The weight of the filter and/or HME device can cause torquing, “kinking,” or obstruction of the circuit and/or the endotracheal tube, or the circuit can be dislodged so that a patient connected to the circuit may not receive the intended gas flow. Moreover, sudden and abrupt movement of the filter may cause the endotracheal tube to injure the patient's airway.

Referring for example to FIG. 1A, a filter and the distal end of a prior art breathing circuit is illustrated, showing a filter 10 that connects to the distal end of a breathing conduit 20. An inspiratory gas conduit 30 provides fresh and recycled gases at outlet 40 into the proximal end 12 of filter 10. The fresh gas inlet port 750 is at the carbon dioxide canister 600, which connects with the distal end of conduit 700 that carries fresh gases from fresh gas source 800. Thus, the entire volume of filter 10 along with the breathing conduits and the canister between the fresh gas outlet into the canister 600 act as a fresh gas mixing space and/or volume. In other words, fresh gas mixing space in a circuit is defined as the space between the fresh gas outlet into a circuit and the distal end of the circuit that leads to a patient airway device. This is illustrated in FIG. 1A, in which the distal end 14 of filter 10 is directly connected to a patient airway device, for example, a breathing mask 50 creating substantial obstruction near a patient's face. In addition to the large fresh gas mixing space between the fresh gas outlet into the circuit, the circuit arrangement in FIG. 1 also suffers from torquing and/or other disadvantages mentioned above. A prior art multilumen circuit, such as the coaxial circuit shown in FIG. 1B, can also be connected to a filter, but suffers the same disadvantages.

Recently, a small catheter mounting tube has become available to minimize the above inconveniences. However, many health care practitioners are concerned with the extra dead space added by the catheter mounting tube and/or the dead space in the respiratory conduit, which is in addition to the dead space produced by the bulky filter. To avoid the obstruction of the filter at the patient face, many practitioners administering anesthesia with a circle system connect filters at the proximal or machine end of the respiratory tubing carrying gases to and from a patient. So, in traditional dual limb circle systems, two filters are necessary, i.e., one filter for each limb respectively.

The Universal F2® system, manufactured by King Systems Corporation of Indiana, includes a unilimb coaxial respiratory conduit with a mating coaxial filter that enables the use of one unitary filter device instead of two filters in independent housings. The coaxial filter can be connected at the proximal end of the multilumen respiratory conduit to provide independent filtration of opposing independent gas flows while connecting the patient respiratory conduit flow paths to the inspiratory and expiratory gas ports on the machine. The Universal F2® system provides tremendous improvements in respiratory care, in part by permitting ready connection and disconnection of breathing conduits and filters to a ventilator or anesthesia machine, while ensuring that the risk of accidental and undetected disconnection or blockage of the inspiratory gas flow is minimized.

The Universal F2® system permits reuse of the proximal terminal, which was disposed of in prior unilimb circuits. A novel multilumen proximal fitting is preferably used to connect a multilumen respiratory conduit to the proximal terminal. The multilumen fitting may incorporate filters in its lumens or be connected to one or more filtered lumens. The Universal F2® system permits ready sterilization or disposal of the flexible conduits carrying gases between a patient and a ventilator or anesthesia machine. Nevertheless, the filter(s) and all tubing and devices distal thereof (i.e., distal tubing and components are on the patient side of the filter(s)), are contaminated by the patient and must be sterilized or disposed of after use.

For decades, typical adult circle breathing circuits (for anesthesia use) and ventilator circuits (for use in an Intensive Care Unit, “ICU”) have been and are still provided in standard lengths of 40, 48, 60 and 72 inches (22 mm ID); standard lengths of 28-30 inches (15 mm ID) have been provided for pediatric use. The use of expandable, pleated tubes (i.e., “flexitube” or “flextube” such as the commercially available Ultra-Flex® by King Systems or Isoflex® by Baxter) provides for a greater range of breathing circuit dimensions, but such adjustable tubes are usually made to conform to the above lengths when expanded, and the entire circuit is disposed of after a single use.

Commonly used devices for use in assisted ventilation derived from the T-piece breathing tube concept. Mapleson described and analyzed different semi-closed anesthetic systems, referred to as Mapleson A-F systems. Although the components and their arrangements are simple, the functional analysis can be very complex as can be seen, for example, in some of the documents referenced herein. The most widely available circuits are those based on the Mapleson D and F type systems, which are known commercially as the Bain circuit and the Jackson-Rees circuit, or modifications thereof. Information on the above circuits can be found in product information associated with devices sold by companies such as Hudson RCI of Temecula, Calif., Intersurgical, Inc. of England, Portex, Inc., of New Hampshire, and King Systems Corporation of Indiana among many others. These prior art circuits are formed of flexible corrugated tubing with a predetermined length. For example, the pediatric CPRAM® circuit is about 50 cm long, and the coaxial Bain circuit is about 180 cm long. In addition, reservoir bags forming part of the circuit are for single use too. The corrugated tubing, unlike flexitube, cannot be axially extended or compressed to a new self-maintained length. The entire bulky and expensive circuit is disposed of after use.

### Safety Has a High Cost in Materials and Pollution

The safety of patients is the foremost concern of healthcare practitioners. The role of respiratory equipment as a source of cross infection leading to respiratory diseases is well known. With the increasing threat of infectious diseases, such as SARS, hepatitis, tuberculosis, and HIV, the need to protect respiratory equipment to minimize exposure of patients to infectious respiratory secretions is more compelling than ever. Disposable devices, including breathing circuits and filters, have been widely used to reduce the chance of passing infectious agents between patients. However, the large and ever increasing amounts of medical waste pose serious problems, such as potential toxic environmental effects caused by its disposal and the costs of providing the disposable components. To the extent contaminated equipment can be sterilized for reuse, there are associated high costs for labor, equipment, cleaning supplies, and storage. Therefore, there is a need for assisted ventilation systems that protect the patient from cross-contamination, yet reduce medical waste and/or the amount of components that are used for a single use before disposal or sterilization.

According to the American Society of Anesthesiologists, there are about 40 million anesthetic cases in the United States annually. In addition, a significantly large number of trauma patients are admitted to emergency rooms and to intensive care units (ICU) that receive assisted ventilation using breathing circuits. The large number of patients using disposable breathing circuits generates a tremendous amount of medical waste. Hauling and disposing of medical waste, particularly transport and disposal outside of urban areas is very expensive. Therefore, there is a compelling need to minimize the amount of plastic and other materials used and disposed of while protecting patients from cross-infection. There is also a need for simple, efficient and convenient resuscitation and assisted ventilation devices that serve multiple functions yet protect the patient as well or better than prior art devices while being more economical to use.

### SUMMARY OF THE INVENTION

For the purpose of describing the present inventions, certain definitions are provided herein. Fresh gas mixing space or mixing space refers to the space or volume between the fresh gas outlet into a circuit or breathing device (e.g., filter and/or HME) and the distal end of the distal breathing tube thereof, wherein the distal end of the distal breathing tube can be connected to a patient or patient airway device. The fresh gas port or fresh gas flow outlet refers to the distal end of the conduit from the fresh gas source (i.e., anesthesia machine, ventilator and the like) from which only fresh gases are emitted into a circuit or device that can be connected at its distal end to a patient airway device. The fresh gases are in contrast to the refresh(ed) gases that are recirculated and pass through the carbon dioxide absorber canister. In certain embodiments of the present invention, the fresh gas outlet is integral to and opens into a distal conduit (or a distal fitting thereof that can be connected to a patient airway device. In other embodiments of the present invention, the fresh outlet is located internally of a distal conduit (or a distal fitting thereof) that can be connected a patient airway device. For ease of description, references to the distance between the fresh gas outlet and the distal end of the distal conduit will include a distal fitting thereof unless otherwise indicated.

Fm is defined as the concentration of anesthetic gas delivered by an anesthesia machine (i.e., the concentration of gas that is specified according to the anesthesia machine control). Fp is defined as the concentration of anesthetic gas actually inhaled by the patient (i.e., the concentration of gas that can be measured by monitoring the gas that is inhaled by the patient). It is desirable for the deviation between Fm and Fp to be small and predictable, because an anesthesiologist needs to know the concentration of anesthetic gases inhaled by their patient. If the deviation between Fm and Fp is large, either (1) the patient is receiving a smaller concentration of anesthetic than desired (i.e., is underanesthetized), or (2) the patient is receiving a higher concentration of anesthetic than desired (i.e., is overanesthetized). Both of these cases are dangerous.

The larger the distance between the fresh gas outlet into a circuit and the patient, the greater the deviation between Fm and Fp. This is because the smaller this distance, the less space there is for the fresh gas to be diluted by mixing with other gases in the circuit. Therefore, the closer the fresh gas port is to the patient, the smaller the deviation between Fm and Fp, and thus it is desirable and useful for the fresh gas outlet to be as close as possible to the patient.

When a first component “A” is distal to a second component “B”, A is located in a position that is further from the anesthesia machine than B and A is closer to the patient than B. Thus, if the filter housing is distal to the fresh gas outlet, then the filter adds mixing space between the fresh gas outlet and the patient, and consequently it is expected that the deviation between Fm and Fp will increase. Prior art filters and their housings have been located distal to the fresh gas outlet. In contrast, in the present inventions, the fresh gas port is distal to the filter medium. In an embodiment of the present invention, the mixing space between the distal end of a breathing circuit or other breathing device and the fresh gas outlet therein is less than about 15 cc, and in a preferred embodiment the mixing space is less than about 5 cc.

In another aspect, the present invention involves a novel breathing system that has a greater reusable portion than prior art circuits. Hence a smaller amount of the breathing circuit together with a disposable filter is disposed of after use by a patient, leading to reduced supply costs and reduced medical wastes, yet improving or maintaining patient safety. In a preferred embodiment, a breathing circuit with substantially no mixing space and/or resistance to spontaneous breathing has a smaller portion that is disposable and a larger portion that is reusable than in prior art circuits, and particularly so in comparison to prior art circuits of about the same length.

In an embodiment, the patient or distal end of a breathing circuit or device has a small conduit and/or filter portion that is disposable, referred to as a “distal disposable breathing device” or “distal disposable filter and tube device”, while the proximal or machine portion is reusable. For the sake of convenience, a distal disposable filter and tube device conforming to a preferred embodiment of the present invention is referred to as the F-tube™. In an embodiment, the filter and the tubing are bonded and integrally constructed. The length of the tubing in the distal disposable breathing device is long enough to keep a filter or other device connected thereto sufficiently far away from the patient's face so as not to interfere with medical care being provided to the patient, yet short enough to reduce the amount of material that is contaminated by a patient that requires disposal or sterilization. In embodiments of the present inventions, the length of the distal disposable breathing device is between about 10 cm and about 50 cm, between about 15 cm and about 40 cm, and between about 20 cm and about 30 cm.

In a preferred embodiment, a fresh gas flow outlet is provided near or at the distal terminus of the distal disposable breathing device, therefore substantially eliminating mixing space and/or volume, thus providing for a minimal deviation between Fm and Fp, and further providing for an increase in the inspired/delivered (FI/FD) ratio of anesthetic gases to achieve a more accurate inspired gas concentration. For the purposes of the present inventions, by substantially eliminating mixing space, it is meant that mixing space in a breathing device or circuit created between the fresh gas distal outlet into a breathing device or circuit and the distal end of a breathing device or circuit is less than about 5 cm³. Fresh gases refer to the gases provided directly from the fresh gas source, for example an anesthesia machine. “Refresh gases”, “refreshed gases” or “recirculated gas” refer to the gases coming from the CO₂ absorber canister.

An alternative embodiment of the F-tube™ device includes an adjustable length distal breathing tube (e.g., flexitube), which places a patient airway device in fluid communication with the proximal portion of a circuit via a filter. Preferably, the filter and tube are bonded together to form an integral device. The proximal portion of a breathing circuit that incorporates an F-tube™ may optionally include an adjustable length proximal tube that permits further adjustment of the volume in the circuit.

In contrast to the prior art, the filter in breathing circuit embodiments of the present inventions is located neither at the distal end or the proximal end of the breathing circuit. The filter in the present inventions is located at a point between the distal and proximal end of the breathing circuit to minimize medical waste while maintaining patient safety and further being effective and practical. A preferred distance between the filter and the distal end of the distal disposable breathing device is between about 10 cm and about 50 cm.

An intermediate circuit fitting of the present invention permits ready connection and disconnection of the distal disposable filter device of the present invention to reusable circuit components of the present invention.

In an embodiment, a distal filter device (i.e., a filter and distal breathing conduit used at the patient side of the breathing system) has substantially no mixing space by having a fresh gas flow outlet near to or at the distal terminus of the distal filter device, wherein the distal terminus can be connected to a patient airway device.

These and other advantages of the present invention and its various embodiments are more fully described below with reference to the following drawings.

### DESCRIPTION OF THE DRAWINGS

In referring to the following figures, it should be understood that the drawings are made to facilitate understanding of the present invention, and therefore, as one of skill in the art will immediately recognize, parts may be out of proportion or in different positions with respect to one another than in actual practice. Hence, one of skill in the art will understand that part dimensions, fittings and connections will accommodate desired inspiratory and expiratory functions, with consideration given, for example, to whether a conduit is a gas delivery conduit or expiratory conduit. Preferably, devices of the present inventions will be constructed so as to allow easy spontaneous ventilation and have flow resistance less than about 1 cm H₂O pressure drop at 10 L/min.

FIG. 1A illustrates portions of a prior art breathing circuit, wherein a filter is located at the far distal end or patient end. Note that the fresh gas outlet into the circuit is located at the carbon dioxide canister, far remote from the patient, creating a large mixing space.

FIG. 1B illustrates that a coaxial circuit can be used to carry gases to and from the filter in 1A, but will also create a circuit with a large mixing space.

FIGS. 2A-D illustrate kit components of the present invention and a breathing device of the present invention incorporating the kit components. FIG. 2A is a top plan view of an embodiment of a disposable distal filter device of the present invention, having a housing forming a large filtered lumen or conduit and two other lumens or conduits. FIG. 2B is a side perspective view of a reusable fresh gas flow (FGF) delivery fitting, i.e., an intermediate circuit fitting or interface for connecting a disposable distal breathing device of the present invention to form a breathing circuit in an assisted ventilation or anesthesia system. FIG. 2C illustrates a partially exploded plan view of a breathing circuit constructed in accordance with the present invention, including an optional integral intermediate circuit fitting on the distal end of the reusable portion. FIG. 2D illustrates an alternative construction, including an optional FGF delivery fitting or adaptor located between the second breathing tube and the proximal Y connector. A second, side view of the filter housing is shown in alignment with its location in the circuit shown in FIG. 2D.

FIG. 3 is a partial cut-away view of the distal portion of a breathing circuit of the present invention showing a distal disposable filter device of the present invention connected at its proximal end to the distal end of a reusable proximal breathing tube 240.

FIGS. 4A-C illustrates the versatility of a disposable F-tube™ device of the present invention. FIG. 4A illustrates how a single F-tube™ device can be detachably connected to a portable assisted ventilation system, while FIGS. 4B and 4C illustrate how the same F-tube™ device can then be subsequently and/or alternatively used in an operating room to connect a patient to an anesthesia machine, and then in an ICU to connect the patient to an assisted ventilation machine.

FIG. 5 illustrates an alternative embodiment of a novel distal disposable filtration device, including a housing containing a filter medium, and further including a distal fresh gas flow inlet and outlet that permits fresh gases to be provided distal of the filter.

FIG. 6 illustrates an alternative embodiment of a novel distal disposable filtration device, including a filter for filtering gases entering and leaving the filter housing and a distal fresh gas flow inlet and outlet on a distal fitting at the distal end of an adjustable length distal tube. The fresh gas flow inlet line is connected at an angle on the distal fitting to project away from a patient during use.

FIG. 7 illustrates an alternative embodiment of the device in FIG. 6, in which a fresh gas flow inlet is located distal of but close to the distal filter housing, and a fresh gas flow conduit extends internally from the fresh gas flow inlet so that the fresh gas flow outlet is at the distal end of the distal breathing conduit, minimizing mixing space, while reducing the dimensions of the circuit.

FIG. 8 illustrates an alternative embodiment of the device of FIG. 7 in which the fresh gas flow-connecting conduit is integrally formed into the filter-housing wall.

FIG. 9A illustrates a partial cut-away and exploded top plan view of a multilumen filter embodiment in which both the inspiratory and expiratory flow paths are filtered in the circle system.

FIG. 9B is a side view of the device in FIG. 9A.

FIG. 10A illustrates a partial cut-away and exploded top view of an alternative breathing device with a multilumen filter embodiment.

FIG. 10B is a side view of the device in FIG. 10A.

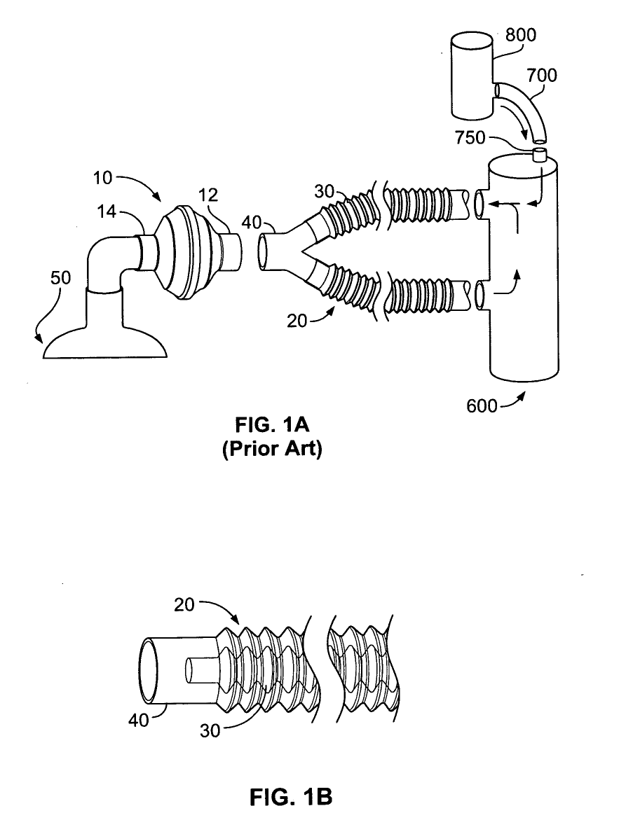

### FURTHER DETAILED DESCRIPTION OF THE INVENTION

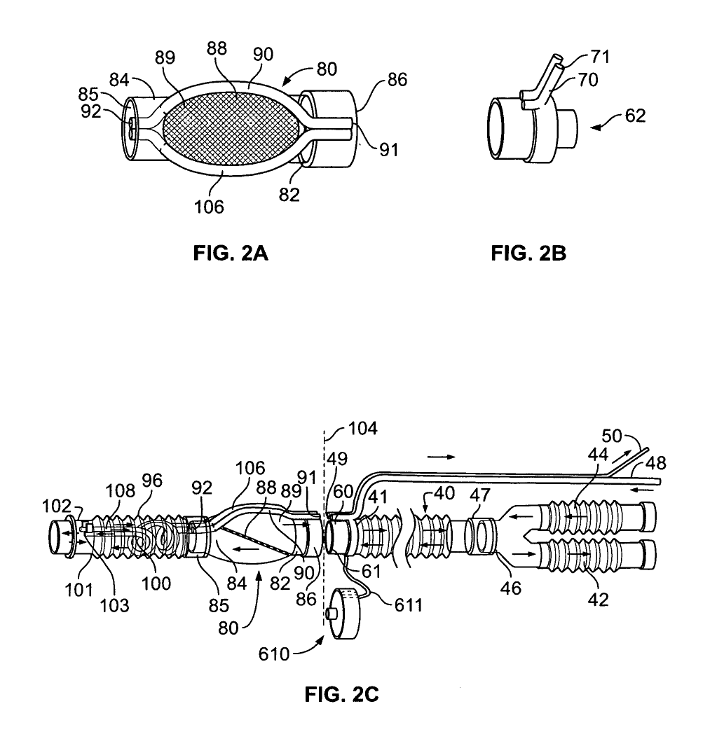

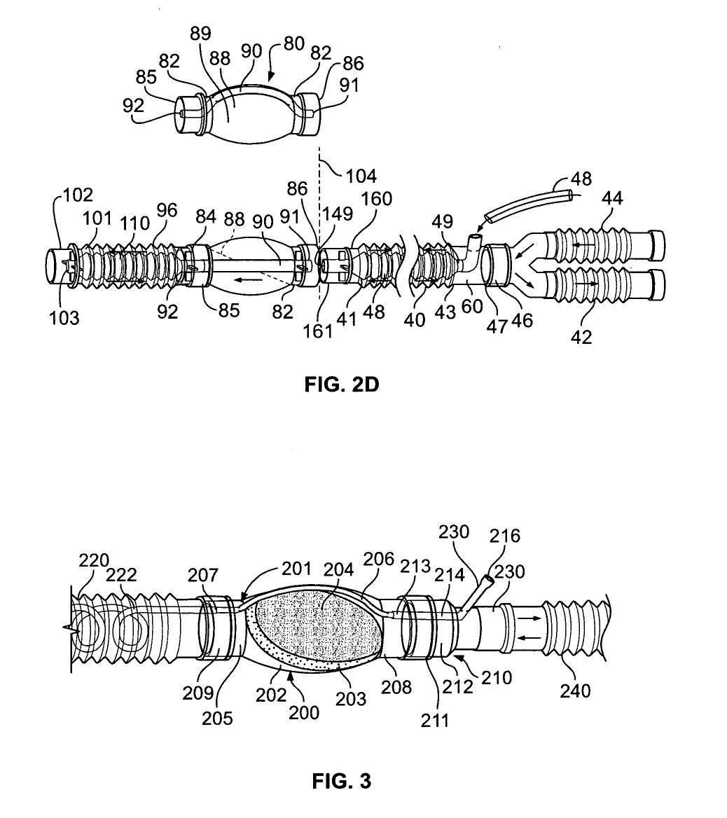

Referring to FIGS. 2A-D, a distal disposable filter and tube device in accordance with the present invention and components for forming same is illustrated; note that the fresh gas outlet is located at the distal terminus of the device. Use of this distal breathing device will lead to substantial reduction of medical wastes. This benefit of the present inventions is highlighted by showing reusable components to the right side of the dotted line 104 while disposable components are shown to the left side of dotted line 104. By disposable, it is to be understood that the components are designed for use by a single patient prior to disposal. If the costs of sterilization make it practical, it is contemplated that after sterilization disposable components might be reused. The circuit of FIGS. 2C-D comprises an optional proximal breathing (or “rebreathing”) conduit 40, which connects to a Y connector and branches into an exhaust conduit 42 and a recirculated gas conduit 44. The Y connector can be a T connector that functions similarly. Exhaled gases can be exhausted from tube 42 and recirculated via tube 44 following CO₂ removal. Proximal rebreathing conduit 40 is preferably formed of flexitube that enables continuous adjustment of the respiratory gas volume therein for each patient, and allows for optimizing the inspired/delivered gas concentration ratios (i.e., FI/FD ratios) to a target level. Surprising and novel methods and devices for providing safe anesthesia while saving substantial amounts of anesthetic gases in comparison to prior art methods and devices that increase the FI/FD ratios are taught in copending U.S. patent application Ser. No. 10/777,772, Ser. No. 10/254,700 and Ser. No. 10/390,070. Conduits 42 and 44 may be separate tubes connected by Y-fitting 46, or may be a single tube having a dividing wall. Specifically incorporated by reference as if reproduced in full below are the full disclosures of copending U.S. patent application Ser. No. 10/777,772, Ser. No. 10/254,700 and Ser. No. 10/390,070.

A proximal fresh gas tube 48 can provide fresh gases from the source in a continuous manner to the fresh gas outlet 103 located near the patient via a first, fresh gas connecting outlet 49 near the distal end 41 of proximal breathing tube 40 and thereafter to the second fresh gas outlet 103 at distal end 101. An optional proximal gas monitor line 50 can be provided as well. Tube 48 and line 50 will be discussed in more detail below in connection with other components.

### New Intermediate Circuit Multilumen Fitting and FGF Delivery Adaptor

A novel intermediate circuit multilumen fitting 60, or “fresh gas flow (FGF) delivery adaptor or fitting”, can be connected to the distal end 41 of proximal breathing tube 40. The intermediate multilumen fitting 60 can be integral with tube 40 or be separately provided as with fitting 62 shown in FIG. 2B. Thus, fitting 60 or 62 can serve as a distal interface between disposable and reusable components in a breathing circuit, such as that shown in FIG. 2C. In the alternative embodiment shown in FIG. 2D, a fitting such as one of fittings 60 or 62 can be directly connected to the distal end 47 of Y-connector 46 that is connected with conduits 42 and 44, or fitting 60 may be connected to a unilimb circuit such as a Universal F2® circuit. Fresh gas outlet 49 is configured to readily connect to a proximal fresh gas line 90 and thereafter to fresh gas conduit 100. The distal portion of proximal fresh gas tube 48 can be integrally formed into fitting 60. In the alternative, a suitable proximal fresh gas connector conduit 70, such as shown in FIG. 2B, can be provided in an intermediate circuit fitting, such as 62, which has a proximal socket 71, male or female, for connection to the distal end of a proximal fresh gas tube 48. It should be clear from the foregoing that, prior to the present invention, such a distal filter device that acts as an interface between disposable and reusable components in a breathing circuit and reduces medical wastes did not exist. Further, in an embodiment, a FGF delivery adaptor or a disposable intermediate breathing circuit fitting is provided which enables connection of a reusable breathing conduit to a distal disposable filter device that maintains a filter at a desired distance from a patient, preferably while permitting little or substantially no mixing space.

### Distal Disposable Filter Device and Circuit Using Same

FIGS. 2A, 2C and 2D illustrate a novel distal disposable filter device of the present invention, with FIG. 2C showing the device connected into a circuit shown in partial exploded cut-away view. A housing 80 includes a filter chamber 89 between a proximal conduit 82 and a distal conduit 84. The housing of filter 80 has a distal end 85 and a proximal end 86. Proximal end 86 is preferably shaped to mate with the corresponding distal end of an intermediate circuit multilumen fitting of the present invention, such as 60 or 62. A filter medium 88 is preferably situated in a diagonal fashion in filter chamber 89 to maximize surface area while minimizing the radial profile of the housing and providing substantially no resistance to spontaneous breathing. The filter medium permits passage of desired gases while blocking particulates and other undesired materials from passing between conduit 82 and conduit 84 or vice versa.

A fresh gas line connection conduit 90 is connected to or forms part of the exterior wall of housing 80. Conduit 90 preferably does not contain a filter as the fresh gases to be carried thereby are provided fresh directly from a fresh gas source. Conduit 90 has a proximal end 91 and a distal end 92. Referring to FIGS. 2A and 2C, proximal end 91 of conduit 90 is shaped to sealably connect to fresh gas outlet 49 while conduit 82 is independently sealably connected to conduit 40 via fitting 60 at distal end 61.

The arrows in FIG. 2C inside of filter chamber 89, tube 96, and proximal conduits 40, 42 and 44 indicate the direction of respiratory gas flow in the circuit during use (i.e., inspiratory and expiratory flows). While conduits having cross-sectional shapes that are substantially circular are shown in the drawings, it is envisioned that a wide variety of cross-sectional lumen shapes can be used, and that different lumens formed in a multilumen conduit, fitting or other device of the present invention may have a wide range of different shapes and sizes. Further, the cross-sectional shape of a lumen may change along its axial length. For example, conduits 82 and 84 and chamber 89 form a lumen or passage that changes in shape along its axis. Further conduits may not be axially linear and can be configured in a wide variety of axial paths, so a lumen may be coiled in a non-limiting example.

As shown in FIG. 2C, in a preferred embodiment the distal end 85 of distal conduit 84 in housing 80 is connected, and preferably sealably fastened or bonded, to a distal breathing tube 96. Distal fresh gas flow tube 100 is connected, and preferably sealably fastened or bonded, to the distal end 92 of fresh gas flow line connection conduit 90. In a preferred embodiment, tube 96 has at least a portion formed of flexible, axially extendable and compressible pleated tubing. Such tubing maintains a minimum radius, yet will also substantially maintain a length and/or angular shape to which it is manipulated. The accordion like pleats permit the tube to expand and contract to a predetermined degree associated with the amplitude of the pleats and the maximum and minimum angle formed by the annular wall portions meeting to form the pleats. Further, at least part of a length of tube 96 can be cylindrical or conical (e.g., be of a smaller diameter at the patient or distal end). Fresh gas flow line 100 can be formed of flexible and coiled tubing, pleated tubing, or be a suave™ tube. The distal end fresh gas port 103 of line 100 can be connected to or connected proximate to the distal fitting 102 on tube 96, which enables the device to deliver fresh gases from line 100 to the fresh gas outlet 103 at the distal end 101 of breathing conduit 96 without any substantial gas mixing or dilution of the fresh gases (i.e., deviation between Fm and Fp is small at clinical flows).

In FIG. 2D, the FGF adaptor or fitting 60 is located at the proximal end of the second proximal tube 40. Distal end 149 of conduit 48, which can be a pleated tube, is located within pleated tube 40. Distal end 149 of conduit 48 and distal end 161 of fitting 160 connect with respective proximal ends 91 and 86 of the lumens in housing 80. Preferably, housing 80 comprises a FGF conduit 90, which can connect at its distal end to tube 110, which can be a pleated tube as well. Tube 110 has a fresh gas outlet 103 located at the distal end of tube 96 or distal fitting 102. The distal ends 103 and 101 of tubes 100 and 96 are preferably bonded to a common distal fitting. The distal fitting preferably has flanges or a perforated annular disk to connect the distal ends of the two tubes and/or means to prevent blockage of inspiratory/expiratory gases during spontaneous or assisted ventilation.

The length of a pleated tube used for line 110 and conduit 48 can be several inches or pleats longer, than the maximum expanded length of tube 96 or tube 40, respectively so that manipulation is facilitated while disconnections are avoided even if axial extension or contraction is made. Hence, when the distal ends of line 110 and tube 96 are connected to a common distal fitting, the proximal end of line 110 is connected to the distal end 92 of conduit 90 in housing 80. Likewise, use of pleated tubing for conduit 48 that is slightly longer than the maximum expanded length of tube 40 will minimize disconnection risk at either its distal or proximal end connection points when tube 40 is extended or contracted.

The circuit arrangements shown in FIGS. 2C and 2D enable significant anesthetic gas savings when used with the gas-saving methods taught by Fukunaga et al. in the aforementioned patent filings. Furthermore, the circuit maintains minimal or substantially no mixing space, even if the length of tube 96 is adjusted (i.e., expanded or contracted). Either or both proximal conduit 40 and distal conduit 96 can be formed of flexitube that permits continuous adjustment of the volume therein for each patient to enable anesthetic gas savings by optimizing the inspired fresh gas concentration ratios (i.e., FI/FD ratios). Such volume adjustments are possible by expanding or contracting the pleated tubes of conduit 40 and/or 96, whose inner and outer tubes are connected to fittings at their distal and proximal ends that enable mutually axial interaction of the circuit members.

In a preferred embodiment, a disposable kit formed substantially of the components to the left of dotted vertical line 104, can be provided to care providers to replace components disposed of after use by a single patient. Gas sampling line connection conduit 106 and gas sampling line 108 are optionally included. Conduit 106 can be parallel to conduit 90 and can be integrally formed into housing 80. Conduit 106 can be connected to proximal sampling line 50 via intermediate circuit multilumen fitting 60.

Referring further to FIGS. 2B-C, an optional cap 610, illustrated as being formed of substantially transparent material, is provided for protecting the reusable portion of a circuit when not in use. The cap is preferably located at or near the distal end of breathing tube 40. A string 611 is provided, and can be attached to the reusable portion of a circuit, or to a reusable intermediate circuit fitting of the present invention, to ensure that the cap is readily available.

Other structural variations within the scope of the present invention are envisioned. For example, with reference to FIG. 3, distal disposable filtration device 200 comprises a housing 201 that includes a central filter chamber 202 with a filter medium 204. An HME 203 can be combined with filter medium 204 and is connected at an oblique angle to the interior wall of the filter chamber to maximize filter surface area while minimizing the required radial size of the housing needed to meet flow requirements. Extending distally from central filter chamber 202 is a distal breathing conduit 205 that has a distal end 209. Extending proximally from central filter chamber 202 is a proximal breathing conduit 208 that has a proximal end 211. Housing 201 also includes an independent fresh gas flow connecting conduit 206 that has a distal end 207 and a proximal end 213.

An intermediate distal multilumen connector fitting 210 includes a main breathing connecting conduit 212 and a proximal fresh gas flow connecting conduit 214, the distal ends of which are shown detachably connected respectively to the proximal end 211 of proximal breathing conduit 208 and to the proximal end 213 of fresh gas flow connecting conduit 206. Fresh gas flow inlet 216 on the proximal end of proximal fresh gas connecting conduit 214 can be connected to a fresh gas flow line (not shown) and the fitting 210 can be reused without disconnecting same. Intermediate fitting 212 can also be integral with proximal conduit 208 and conduit 206, in which case it will be disposed therewith.

A distal breathing tube 220 is connected to the distal end 209 of distal breathing conduit 205 of filter housing 201. Distal breathing tube 220 may be formed of adjustable length pleated tubing, and is preferably sealably fastened or bonded at its proximal end to the distal end 209 of distal breathing conduit 205. The proximal end of fresh gas flow tube 222 is connected, and preferably sealably fastened or bonded, to the distal end 207 of fresh gas flow connecting conduit 206. The distal port or outlet of conduit 222 (not shown) is near the patient. Fresh gas connecting conduit 206 does not contain a filter medium, and thus substantially laminar flow is possible. Further, as no filter is located in conduit 206, the multilumen housing 201 can be smaller in size than a multilumen filter housing wherein both conduits are filtered. The length of distal tube 220 is preferably long enough to keep the filter sufficiently far away from the patient so as not to interfere with access to the patient by caregivers, yet sufficiently short to reduce medical wastes significantly in comparison to even the most efficient systems currently known, specifically the Universal F2® and the more recent F3™.

The distal end of intermediate distal multilumen connector fitting 210 can be detachably connected to the proximal end of housing 201 so that lumens in conduits 212, 208, 205 and 220 and filter chamber 202 form an uninterrupted flow path that is independent of a fresh gas flow path formed by the lumens in conduits 214, 206 and 222. The intermediate distal multilumen connector fitting 210, filter housing 201 and tubing 220 and 222 are easy to manufacture of medical grade plastic, and/or existing medical tubing and filter components can be modified. While intermediate distal multilumen connector fitting 210 is described in this preferred embodiment as being detachably connected to housing 201, it is envisioned that it may be integrally attached and/or bonded, so that the component 210 forms part of disposable device 200; in that case, users would need to separately connect a fresh gas flow line to inlet 216 and a proximal conduit 240 to the proximal end 230 of intermediate distal multilumen connector fitting 210 whenever device 200 is replaced. Inlet 216 may be provided with a cap (not shown).

Preferably however, filter medium 204 prevents contamination of fitting 210, and therefore fitting 210 and components proximal thereof can be used for different patients without requiring sterilization in between. Components detachably connected at the distal end of intermediate distal multilumen connector fitting 210 can be disposed of between patients. A preferred embodiment of the F-tube™ device comprises housing 201, filter 204, and distal tubes 220 and 222. Preferably, an F-tube™ device can be connected to a reusable multilumen intermediate circuit fitting, such as fitting 210, in a single step.

In embodiments, distal tube 220 is at least about 15 cm in length, at least 20 cm in length, or about 40 cm in length. An alternative embodiment is made from a length of flexitube that compresses to about 10 cm in length and can be extended up to about 50 cm in length. Another embodiment includes a coiled fresh gas tube formed of medical grade plastic capable of delivering fresh gas flows in sufficient amounts to the first distal breathing tube 220, preferably between about 0.5 L/min to about 60 L/min. Suitable fittings, tubing, and housings are preferably formed of medical grade plastic, such as that used to produce commercially available circuits and components.

The F-tube™ is very practical and convenient to use, while minimizing the amount of plastic and other materials manufactured into product, stored in inventory, purchased, shipped and ultimately disposed of after use. For example, in an embodiment only about 25% of the required standard respiratory conduit to form a circuit is disposed of and the rest may be reused. Thus, the reusable components of the circuit can be made of material that is more durable than conventionally used, or the reusable components can be semi-disposable. For example, the reusable components of the breathing circuit (i.e., proximal breathing conduit) can be made of silicone rubber that can withstand multiple sterilization procedures. The reusable conduit can be used numerous times before disposal.

For easy handling in the operating room, a set-up kit can be provided with one set of reusable components and multiple disposable F-tube™ devices (preferably about 5). A benefit to having a multilumen fitting between the disposable components and the reusable components is that it is easy to segregate the medical waste portion from the reusable portion, which may be recycled, and only one component needs to be connected and disconnected to connect the multiple lumens. The components may be color coded to facilitate distinguishing between the disposable components and the reusable ones. By reducing the amount of materials that have to be disposed of after a single use, the amount of toxic materials released into the surrounding community is greatly reduced, particularly where incineration is used for disposal. Further, patients and hospitals benefit as it takes less time to assemble and disassemble respiratory circuits made in accordance with the present invention, which leads to reduced hospital costs. Therefore, the present invention has great immediate benefit.

### F-Tube™ Versatility

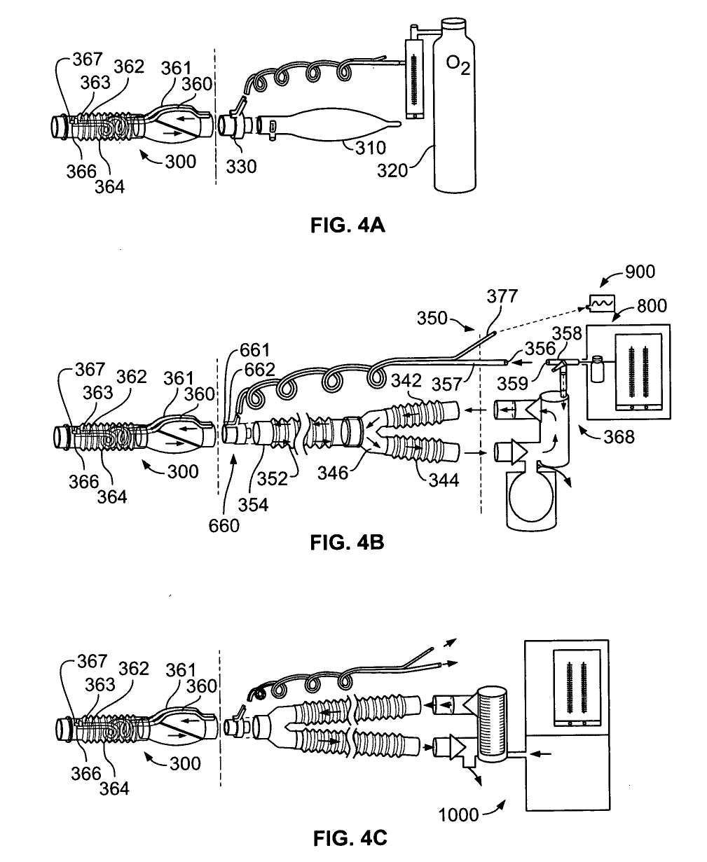

The versatility of an F-tube™ device is illustrated in FIGS. 4A-C. F-tube™ 300 can be connected in an emergency or ambulatory setting to a breathing bag 310 and fresh gas flow source 320 via intermediate circuit fitting 330, as shown in FIG. 4A. A patient involved in an accident or experiencing other trauma may require immediate assisted ventilation. A rescuer that needs to give mouth to mask resuscitation can use the F-tube™ connected to a mouthpiece (not shown). Thereafter the F-tube™ can be connected to a bag and an oxygen source. The paramedics or firefighters can provide manual assisted ventilation during transport and/or in the ambulance or helicopter until reaching the Emergency Medicine Service or Trauma Center, where the F-tube™ can then be connected to the distal end of a standard breathing circuit (dual limb or unilimb).

In an embodiment, an F-tube™ 300 can be disconnected from an ambulatory setting and connected to an operating room anesthesia system 350 as shown in FIG. 4B. A Fukunaga gas saving system is illustrated in FIG. 4B. More details about this system can be found in co-pending U.S. patent application Ser. No. 10/777,772, Ser. No. 10/254,700 and Ser. No. 10/390,070, which, as mentioned above, are specifically incorporated as if reproduced in full herein. Briefly, however, a reusable proximal breathing conduit 352 is detachably connected at its distal end 354 to the proximal end of a F-tube™ device via an intermediate multilumen circuit fitting (i.e., FGF delivery adaptor or fitting). Fresh gas flow inlet 356 on proximal, reusable fresh gas flow line 357 can be connected to an outlet 359 of diverter valve 358 so that fresh gases can be directed through a fresh gas connector 660 and conduit 662 to conduit 360 and to a distal fresh gas tube 362 in distal breathing conduit 364. The fresh gas outlet 366 for distal fresh gas flow tube 362 is located near or at the distal end of the F-tube™. Thus, there is an uninterrupted flow from fresh gas source 800 to outlet 366. Tube 377 running parallel to conduit 357 may be used for gas monitoring which at the distal end 367 is near the patient and at the far proximal end connects to the gas monitoring machine 900 via conduits 361, 363, and 377.

The reusable proximal breathing conduit or second tube 352 is connected at its proximal end to a wye (Y) or T connector 346 that is connected to the carbon dioxide absorber canister in the anesthesia machine 368 inspiratory and expiratory ports. Flows in the distal breathing conduit or first tube 364 are contiguous with flow in tubes 352, 342 and 344. The F-tube™ can be efficiently utilized in such an F-conomy™ system to reduce waste of anesthetic gases. Both the first and the second tube can be readily expanded or contracted to adjust the content of the gases that are breathed to optimize the ratio of the inspired gas concentration in relation to the delivered gas concentration. Further details of gas saving methods involving post-inspiratory valve or distal fresh gas flow input and adjustments of rebreathing conduit volume are contained in the above-mentioned co-pending patent applications.

Following use in the operating room, a patient can be disconnected from the anesthesia machine by disconnecting the F-tube™ 300. The same F-tube™ can then be connected to a ventilator 1000 in the intensive care unit (“ICU”) as shown in FIG. 4C, and/or during transport of the patient by connecting it to an oxygen tank. In an embodiment, an F-tube™ includes three or more conduits, so that at least one conduit 377 can be used for gas monitoring purposes (e.g. O₂, CO₂ monitoring) and/or 357 can be used for pressure monitoring in the ICU, while one conduit serves as a breathing conduit, preferably conduit 364. Because the distal inlet for a sampling line can be placed very close to the patient, monitoring can be done very accurately, and conveniently, while minimizing clutter near the patient's face.

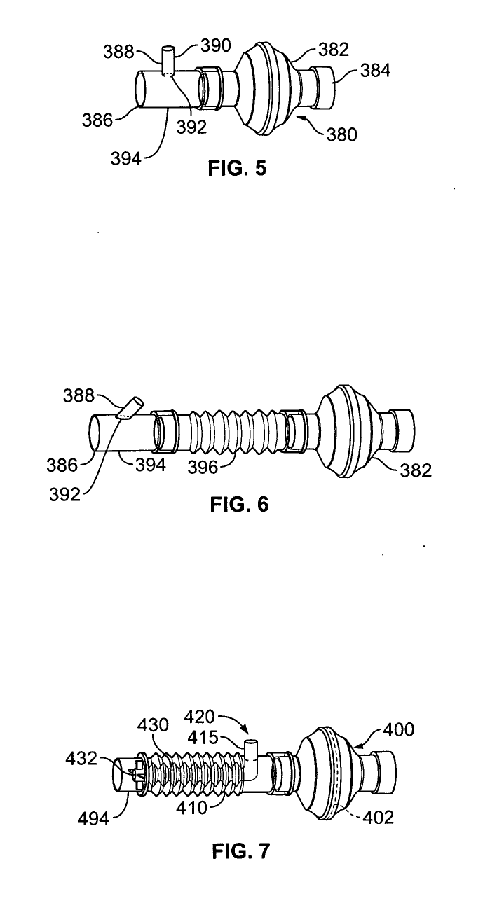

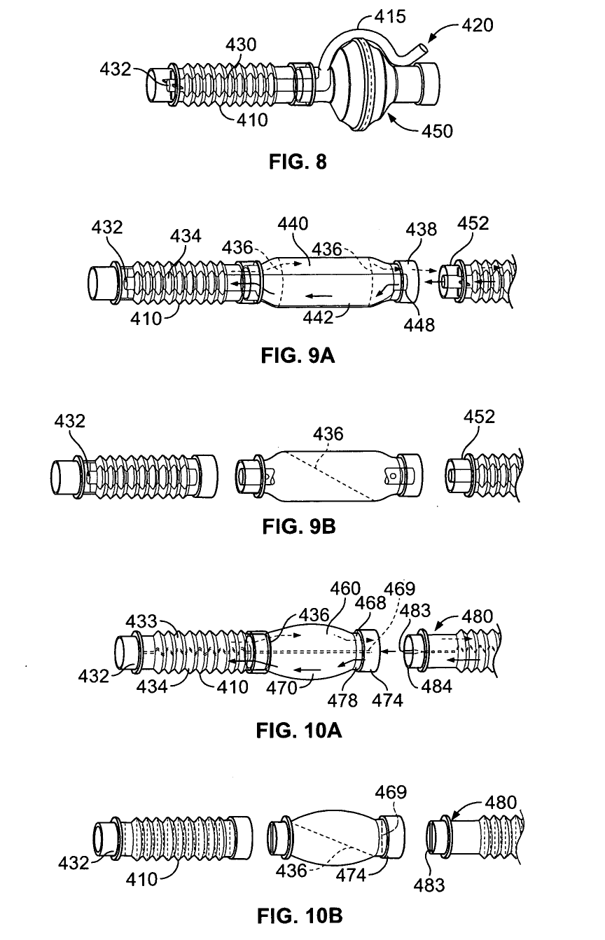

FIGS. 5-10 illustrate components for and alternative embodiments of F-tube™ devices in accordance with the present invention. FIGS. 7-8 demonstrate various ways in which a fresh gas flow inlet can be placed remotely from a distal fresh gas flow outlet, while minimizing mixing space, and reducing the dimensions of the circuit.

With reference to FIG. 5, a filter device 380 includes a filter housing 382 with a proximal fitting 384 and a distal fitting 386 at opposite ends. A fresh gas flow connecting conduit 388 joins distal conduit 394 forming the distal fitting 386. In instances where access to a patient's face is not a concern, device 380 may be connected via distal fitting 386 to a patient airway device and via proximal fitting 384 to a standard breathing circuit. Fresh gases can be provided through inlet 390 to fresh gas connecting conduit 388. Fresh gases in conduit 388 are provided to the distal end of the lumen in fitting 386 via fresh gas outlet or port 392.

FIG. 6 illustrates an alternative embodiment of the device of FIG. 5, which functions similarly. The distal fitting 386 with fresh gas connecting conduit 388 are at the distal end of a distal breathing tube 396 connected at its proximal end to the distal end of filter housing 382. Tube 396 may have a fixed volume, or be formed of flexitube that has an adjustable volume. In embodiments, breathing tube 396 can be provided in various lengths, for example, 10 cm or more in length, 15 cm or more in length, 17 cm or more in length, 20 cm or more in length, or 30 cm or more in length. Preferably, tube 396 is not more than 50 cm in length, even when it is formed of flexitube that is extended to its maximum length.

In FIG. 7, a filter housing 400 containing a filter 402 is connected at its distal end to distal breathing conduit 410. Conduit 410 may be of a predetermined length between about 10 cm and about 50 cm, or in a preferred embodiment is formed of flexitube that has a minimum compressed length of 10 cm and an axially expanded length of about 50 cm. A fresh gas flow connection conduit 415 is integrally formed in the distal portion of filter housing 400. Fresh gases can be carried through inlet 420 to a flexible fresh gas line 430 that is bonded to or integral with conduit 415. Fresh gas line 430 may be formed of pleated tubing, and has its distal fresh gas outlet 432 connected near to the distal end of tube 410 and the distal end of tube 430 may have a common distal fitting 494, so as to be axially compressed and extended with corresponding action of conduit 410. Thus, substantially no mixing space is introduced, yet filter housing 400 can be moved sufficiently far from the patient to improve access. Since only the apparatus in FIG. 7 is disposed of, the remaining circuit components in an assisted ventilation system can be reused.

FIG. 8 illustrates an alternative embodiment of the device of FIG. 7 in which a fresh gas flow connecting conduit 415 is integrally formed onto or into the distal filter housing wall. The inlet 420 for fresh gases is on the proximal end of the filter housing 450, which further removes it from interfering with patient access. However, the fresh gas outlet 432 of the fresh gas line 430 is still maintained close to the patient. Distal end of tubes 430 and 410 can have a common distal end fitting.

Referring to FIGS. 9A-B, a multilumen filter embodiment is illustrated wherein gases passing to and/or from both the coaxial inner and outer tubes of breathing conduit 410 are filtered. Inner tube 434 can carry fresh and/or recycled (i.e., refresh or refreshed) gases to outlet 432. The distal ends of tubes 434 and 410 can have a common distal fitting. A single filter medium 436 is shown situated diagonally in FIG. 9B to cross both filter chambers 440 and 442. The filter can be installed by placing the filter medium between separate components forming the chambers during manufacture, or separate filter media can be used in each chamber Oust the proximal and distal edges of filter medium 436 are shown in FIG. 9A to facilitate understanding of the invention). The proximal ends 438 and 448 of the lumens in the multilumen filter housing can be coaxial to mate to the distal end fitting of a coaxial circuit, such as distal end 452. FIG. 9A illustrates that a multilumen filter does not have to be coaxial to be used in a coaxial circuit. The design of the filter housing and chambers therein can be optimized to minimize the filter housing exterior dimensions, maximize filter surface area in each filter chamber, yet permit sufficient flow to accomplish the anesthetic and/or assisted ventilation needs of the user, e.g., for spontaneous and assisted ventilation.

Referring to FIGS. 10A-B, a multilumen filter embodiment is illustrated wherein the filter housing is divided by a wall. The housing proximal end forms a proximal fitting 474 having a lumen 468 and a lumen 478. The housing includes chamber 460 and chamber 470, and the distal conduit forms lumens 433 and 434. The distal end of lumen 434 forms an outlet 432. A proximal extension of the middle wall 469 can be inserted into slot 483 of fitting 480. The filter 436 is also situated diagonally with respect to the lengthwise axis of the housing. The arrows in FIGS. 9 and 10 illustrate the direction of flow in the devices when used in circuits within a circle system. In this embodiment, it is preferred that the inspiratory and expiratory gases (i.e., fresh and recirculated gases) are both filtered.

### F-Tube™ Advantages

Many advantages arise from the F-tube™:
a) Since the distal, disposable, breathing conduit is much shorter in length than standard prior art breathing conduits, it is more economical to manufacture, to store, to ship and to dispose of.
b) Since the fresh gas outlet or port is very near the patient, deviation of Fm and Fp is minimized, thus concentration of the fresh gases are equal to or about the same as the fresh gas source, which provides a safer and more economical method of delivering anesthesia and/or respiratory care.
c) It is very easy to use.
d) Medical waste and environmental pollution are decreased.
e) Less plastic and other materials are used to manufacture breathing circuits of the present invention in comparison to prior art breathing circuits.
f) Circuit manufacturing is simplified, especially for multilumen conduits.
g) The circuits contain no or minimal mixing space since the fresh gases can be delivered very near the patient.
h) Circuits using the F-tube™ are more versatile than conventional circuits. For example, in an accident or other trauma situation, a patient can be rescued using the F-tube™, which can be used as a conduit for mouth to mask resuscitation; thereafter the same F-tube™ can be connected to a breathing bag or a ventilator in an ambulance, and then connected to an assisted ventilation system in an emergency room, an operating room, recovery room, diagnostic room (e.g., MRI) or ICU in a hospital. Thus, the multipurpose F-tube™ can be seamlessly integrated and used for all phases of patient care involving resuscitation and/or assisted ventilation, whether the patient is at the scene of an accident, at home, at the hospital, during transit, or anywhere an illness or injury strikes.

The foregoing are non-limiting examples of the advantages of the present invention. In view of these advantages, one of skill in the art will desire new devices constructed in accordance with the present invention. In an embodiment, a breathing system can be constructed with a device that comprises at least a first and a second tube and a filter; the filter has a proximal and distal end; the first tube is attached to the filter distal end, and the second tube can be attached to the filter proximal end, wherein the second tube can be detached from the filter for reuse in a breathing circuit formed with the foregoing components. The filter and the first tube may be disposed of after a single use. The first tube has a length sufficient to maintain the filter at a desired distance from a patient airway device, such as an endotracheal tube, when connected thereto. In a preferred embodiment, a fresh gas outlet is provided at the distal end of the first tube resulting in minimal mixing space or substantially no mixing space in the circuit. In a preferred embodiment, the first tube, or disposable distal breathing conduit, is formed of an accordion-like, expandable and compressible pleated tube, e.g., flexitube.

A circuit can be constructed with the present invention in combination with a coaxial filter and a coaxial respiratory conduit such as that in the Universal F2® devices from King Systems Corporation, of Indiana. Universal F2® device components can be used as the second or reusable conduit in the circuit, particularly where it is desired that the distal disposable conduit be short and the proximal conduit be long.

In an alternative embodiment, the distal conduit (i.e., first tube) is a rebreathing tube comprising at least one conduit that is a nonconventional conduit, such as the coiled tube disclosed in U.S. Patent Publication No. 2003/0075176 A1.

### F-Conomy Kit™—Components and Operation

The present invention may be provided in kit form. A set-up kit may be required by a caregiver using the invention for the first time or when reusable components for a circuit are required. For example, the reusable conduit and other reusable components may be included in a first or set-up kit with disposable components, whereas for subsequent uses, the reusable components would not be included in a disposable kit. A preferred kit, referred to herein as the F-conomy Kit™, comprises the F-tube™, a second or proximal rebreathing tube (reusable), and optionally a wye (Y) or T connector (with extension tubes if necessary) and/or a unilimb F2® circuit and components, including a F2® proximal terminal. Other kit components may include a bag, mask, and/or mouthpiece. For example, emergency caregivers may have a customized kit containing the F-tube™, a bag, a mouthpiece and/or mask, while a basic disposable kit in other circumstances may comprise the F-tube™, and optionally other components may be included that a particular procedure is likely to require.

As is clear from the foregoing, the F-tube™ is designed to be a disposable component of a breathing circuit. In summary, a preferred embodiment comprises a filter and preferably a unilimb multilumen distal or first conduit. A first lumen is preferably comprised of flexitube and serves as a rebreathing tube. The second and optional additional lumens are formed by flexitube, coiled tubing, or suave™ tubing. A suave™ tube is radially collapsible and can expand up to a maximum radius under normal respiratory care pressures wherein it has low compliance in the radially outward. The second lumen can carry fresh gases and be connected directly to a fresh gas source in an anesthesia machine. Another lumen can serve to carry gases to a gas monitoring line that connects to a gas monitoring machine. FGF (fresh gas flow) and gas monitoring lines can connect to the filter housing, where they can bypass the filter media in corresponding lumens.

The present pioneer invention has been described with reference to exemplary embodiments only, and incorporates by reference numerous teachings. For example, the invention emphasizes the surprising gas saving (i.e., low flow) techniques recently discovered by Fukunaga et al because of the significant advantages. However, the distal filter and conduit can be used in standard anesthetic techniques and systems, such as the circle system with standard flows and the Mapleson D system with high flows, while minimizing the disposable portions of the circuit(s). Therefore, many variations to the disclosed embodiments are envisioned to be within the teachings and spirit of the present application.

1. A device for filtering and providing patient respiratory gases, comprising:
a housing, said housing comprising at least one wall defining at least one filter chamber between a proximal port and a distal port at opposite ends of said chamber, said chamber having a filter medium that filters flow between said proximal and distal ports,
said device further comprising a first distal conduit comprising a first distal end and a first proximal end,
wherein said filter housing distal port is adapted for connection to said first proximal end of said first distal conduit to pass respiratory gases between said housing and a patient when a patient airway device is connected to said first distal end of said first distal conduit,
said device further comprising a fresh gas outlet located about at said first distal end of said first distal conduit wherein fresh gases may enter said first distal conduit from said fresh gas outlet, wherein the mixing space in said device is less than about 15 cm³.

2. The device of claim 1, wherein said first distal conduit comprises a first pleated axially expandable and compressible tube.

3. The device of claim 1, further comprising a distal fresh gas flow conduit, having a second distal end and a second proximal end, said housing further comprising a fresh gas flow connecting conduit having a third proximal end and a third distal end, wherein said second proximal end of said distal fresh gas flow conduit is connected to said third distal end of said fresh gas flow connecting conduit, said second distal end of said distal fresh gas flow conduit forming or being connected to said fresh gas outlet, wherein said fresh gas flow connecting conduit is connected to the exterior of said housing, is formed in said at least one wall, or passes through said housing.

4. The device of claim 2, further comprising a distal fresh gas flow conduit within or connected to said first distal conduit, said distal fresh gas flow conduit having a second distal end forming or being connected to said fresh gas outlet.

5. The device of claim 4, wherein said distal fresh gas flow conduit comprises a tube selected from the group consisting of a second pleated axially expandable and compressible tube, a coiled tube, and a suave™ tube.

6. The device of claim 5, wherein adjustment of the length of said first distal conduit does not alter the mixing space.

7. The device of claim 1, wherein said first distal conduit has a length selected from the group consisting of at least 20 cm, less than about 50 cm, and greater than about 3 cm and less than about 50 cm.

8. The device of claim 1, having a total length of less than about 50 cm.

9. The device of claim 1, wherein said mixing space is less than about 5 cm³.

10. A breathing circuit having substantially no mixing space and being of a first length, comprising a disposable distal portion and a reusable proximal portion, said disposable distal portion being smaller than is disposable in a prior art circuit when the prior art circuit has a length about equal to said first length.

11. The circuit of claim 10, wherein said disposable distal portion comprises a first distal conduit and said reusable proximal portion comprises a second proximal conduit, said first distal conduit being operatively connectable to said second proximal conduit, wherein said first distal conduit is detachable from said second proximal conduit after use of said circuit by a single patient for disposal or sterilization of said first distal conduit, and wherein said second proximal conduit may be reused.

12. The circuit of claim 11, wherein said first distal conduit is less than about 50 cm in length.

13. The circuit of claim 11, further comprising a filter attachable between said first and second conduits.

14. The circuit of claim 13, wherein said filter forms part of said disposable distal portion and said disposable distal portion has a total length of about 50 cm or less.

15. The circuit of claim 10, wherein said disposable distal portion comprises a first distal conduit and a filter housing, said filter housing comprising at least one filtered conduit, and said reusable proximal portion comprises a second proximal conduit, said first distal conduit being operatively connectable to said second proximal conduit via said filter housing, wherein said first distal conduit and said filter housing is detachable from said second proximal conduit after use of said circuit by a single patient for disposal or sterilization, and wherein said second proximal conduit may be reused.

16. A device for use in a breathing circuit, said device having a machine end and a patient end, comprising:
a housing, said housing comprising at least one wall defining a first lumen, wherein at least a portion of said first lumen forms a filter chamber between a proximal port and a distal port at opposite ends of said chamber, said chamber having a filter medium that filters flow between said proximal and distal ports,
said device further comprising a first distal conduit comprising a first distal end and a first proximal end,
wherein said distal port is adapted for connection to said first proximal end of said first distal conduit to pass respiratory gases between said chamber and a patient when a patient airway device is connected to said first distal end of said first distal conduit,
said housing further comprising a second lumen, said second lumen not containing a filter and useful as a fresh gas conduit to carry fresh gases from said proximal end to said distal end of said device, at least a portion of said fresh gas conduit being integral with said housing.

17. A kit for use in forming an assisted ventilation circuit, comprising at least a first and a second tube and a filter device, said filter device having a proximal end and a distal end, said first tube being attached to said filter device distal end, and said second tube being attached to said filter device proximal end, wherein said second tube may be detached from said filter device for reuse in an assisted ventilation circuit to which it can be attached, and said filter device and said first tube may be disposed of after a single use, at least said first tube creating a rebreathing tube, said first tube having a length sufficient to maintain said filter device at a desired distance from a patient airway device when connected thereto, said kit further comprising a fresh gas connection conduit having an input for connection to a fresh gas source and an outlet in fluid communication with said rebreathing tube, wherein said first tube and said filter device will create substantially no mixing space in a circuit constructed with same.

18. The kit of claim 17, wherein said first tube length is at least 15 cm in length.

19. The kit of claim 17, wherein said first tube length is at least 15 cm and no more than about 50 cm.

20. The kit of claim 17, wherein said first tube is formed of adjustable length pleated tubing.

21. The kit of claim 19, wherein said first tube is formed of adjustable length pleated tubing.

22. The kit of claim 17, further comprising a fresh gas delivery tube connected to said outlet.

23. The kit of claim 22, wherein said fresh gas delivery tube is located inside of said first tube.

24. A method of constructing a breathing circuit, comprising constructing a breathing circuit using the device of claim 1.

25. A method of constructing a breathing circuit, comprising constructing a breathing circuit using the kit of claim 17.

26. The device of claim 1, wherein said fresh gas outlet is connected to a fresh gas flow connecting conduit, said fresh gas flow connecting conduit having a distal end connected to said fresh gas outlet and having a proximal end creating a fresh gas inlet for connection to a source of fresh gases, wherein said fresh gas inlet is located proximal of said filter.

27. An assisted ventilation or anesthesia system, comprising the device of claim 1.

28. An assisted ventilation or anesthesia system, comprising the device of claim 9.

29. An assisted ventilation or anesthesia system, comprising the kit of claim 17.
